(19) United States
(12) Patent Application Publication
(10) Pub. No.: US 2025/0299340 A1
(43) Pub. Date: Sep. 25, 2025

(54) Deep Learning for Four-Dimensional (4D) Modeling of Glioblastoma Multiforme with Tumor Treating Fields (TTFields) Therapy

(71) Applicant: The Regents of The University of California, Oakland, CA (US)

(72) Inventor: Peter D. Chang, Irvine, CA (US)

(73) Assignee: The Regents of The University of California, Oakland, CA (US)

(21) Appl. No.: 19/079,888

(22) Filed: Mar. 14, 2025

Related U.S. Application Data
(60) Provisional application No. 63/568,388, filed on Mar. 21, 2024.

(51) Int. Cl. G06T 7/00; G16H 30/40; G16H 50/20

(52) U.S. Cl. CPC G06T 7/0016; G16H 30/40; G16H 50/20; G06T 2207/10088; G06T 2207/20084; G06T 2207/30016; G06T 2207/30096

(57) ABSTRACT

The technology disclosed relates to deep learning for four-dimensional (4D) modeling of glioblastoma multiforme with tumor treating fields (TTFields) therapy. In particular, the technology disclosed relates to a system comprising memory and a neural network processor. The memory stores input image data characterizing a current spatial distribution of glioblastoma multiforme (GBM). The current spatial distribution of the GBM is detected at a precursor examination of a patient receiving tumor treating fields (TTFields) therapy. The neural network processor, is in communication with the memory, and is configured to cause a neural network to process the input image data and, in response, generate output probability data characterizing a future spatial distribution of the GBM at a follow-up examination of the patient receiving the TTFields therapy. The neural network determines the future spatial distribution based in part on a time interval between the precursor examination and the follow-up examination.

### PRIORITY APPLICATION

This application claims the benefit of U.S. Patent Application No. 63/568,388, entitled “DEEP LEARNING FOR FOUR-DIMENSIONAL (4D) MODELING OF GLIOBLASTOMA MULTIFORME WITH TUMOR TREATING FIELDS (TTFIELDS) THERAPY,” filed on Mar. 21, 2024. The provisional patent application is incorporated by reference for all purposes.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

### DESCRIPTION OF RELATED ART

Glioblastoma (GBM) is the most common type of malignant (cancerous) brain tumor that starts in the brain in adults. Cancer cells in glioblastoma tumors rapidly grow and multiply. Tumor treating fields (also referred to as TTF or TTFields) has emerged as one of the most effective treatment options for the management of glioblastomas (GBMs). Prediction of growth of tumor is critical for effective treatment of a patient.

It is desirable to provide systems and methods that can predict future growth of GBM tumors to improve the treatment outcomes.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 illustrates an example architectural-level schematic of a system that uses a trained machine learning model to predict growth of tumors at a follow-up examination of a patient receiving TTFields therapy.

FIG. 2 presents an example process flow diagram comprising process operations to predict growth of tumors at a follow-up examination of a patient receiving TTFields therapy.

FIG. 3 presents an example high-level architecture to train a machine learning model to predict growth of tumors at a follow-up examination of a patient receiving TTFields therapy.

FIG. 4 is a schematic representation of an encoder-decoder architecture that can be used to implement the machine learning model of FIG. 1.

FIG. 5 shows an overview of an attention mechanism added onto an RNN encoder-decoder architecture.

FIG. 6 is a schematic representation of the calculation of self-attention showing one attention head.

FIG. 7 is a depiction of several attention heads in a Transformer block.

FIG. 8 is an illustration that shows how one can use multiple workers to compute the multi-head attention in parallel, as the respective heads compute their outputs independently of one another.

FIG. 9 is a portrayal of one encoder layer of a Transformer network.

FIG. 10 shows a schematic overview of a Transformer model.

FIGS. 11A and 11B present a depiction of a Vision Transformer (ViT).

FIGS. 12A, 12B, 12C and 12D illustrate a processing flow of the Vision Transformer (ViT).

FIG. 13 shows example software code that implements a Transformer block.

FIG. 14 shows an example computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The following detailed description is made with reference to the figures. Example implementations are described to illustrate the technology disclosed, not to limit its scope, which is defined by the claims. Those of ordinary skill in the art will recognize a variety of equivalent variations on the description that follows. Reference will now be made in detail to the exemplary implementations of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the apparatuses, devices, systems, and methods described herein. None of the features or components shown in the drawings or discussed below should be taken as mandatory for any specific implementation of any of these devices, systems, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general-purpose signal processor or a block of random-access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

### INTRODUCTION

Glioblastoma (GBM) is the most common type of malignant (cancerous) brain tumor that starts in the brain in adults. Cancer cells in glioblastoma tumors rapidly grow and multiply. Glioblastoma is a devastating type of cancer that can result in death in fewer than six months without treatment. It is important to seek diagnosis and treatment as soon as possible to prolong a patient's life. Glioblastoma accounts for almost half of all cancerous brain tumors.

Prediction of future tumor growth and recurrence are critical in the management of patients with glioblastoma multiforme (GBM). Glioblastoma treatments include radiation therapy, intensity-modulated radiation therapy, stereotactic radiosurgery, chemotherapy, immunotherapy, tumor treatment fields (TTF), etc. Tumor treating fields (also referred to as TTF or TTFields) is a noninvasive and innovative therapeutic approach. TTF has emerged as one of the most effective treatment options for the management of glioblastomas (GBMs). TTF is administered by delivering low-intensity, intermediate-frequency, alternating electric fields to human GBM function through different mechanisms of action. The use of TTF inhibits mitosis and the cell cycle, induces cancer cell autophagy, disturbs DNA repair, undermines cell migration, and thus suppresses tumor growth and invasion.

The technology disclosed comprises systems and methods for estimating future areas of GBM spread across multiple timepoints using MRI in patients receiving tumor treating fields (TTFields) therapy. The technology disclosed uses deep learning techniques for estimating future areas of GBM spread.

In one implementation, to generate training data, a single institutional database is queried to identify adult patients with histologically confirmed newly diagnosed and/or recurrent GBM undergoing TTFields therapy. For newly diagnosed GBM, patients received TTFields with temozolomide after maximal debulking surgery and chemoradiation therapy. For recurrent GBM, patients received TTFields as monotherapy. For each patient, all serial follow-up MRI exams were obtained, including T1 (pre-/post-contrast), T2, and T2/FLAIR sequences. On each exam, all regions of enhancing tumor core (excluding peritumoral edema, necrotic core, or resection cavity) were delineated and aligned across time points using nonlinear deformable registration.

For any given pair of serial exams, a convolutional neural network (CNN) is trained to predict future GBM tumor on the follow-up examination given the precursor examination and time interval between the studies (i.e., the precursor examination and the follow-up examination). The model is implemented as a three-dimensional (3D) encoder-decoder architecture yielding a dense per-voxel prediction of future tumor probability optimized using a binary cross-entropy loss function. Class weights are used to develop high sensitivity and high positive predictive value (PPV) model variants. To generate final logit scores, time information is concatenated to the penultimate feature map as an additional feature channel, allowing the model to calibrate each estimate based on elapsed time between any pair of exams. Upon convergence, a 4D learned representation allows for prediction of spatial distribution of GBM tumor at any future time point.

In one instance, a total of 123 patients (1112 total MR exams) were identified. For any given single patient, a median of 6 follow-up exams (IQR 2.5-12.5) at a median interval follow-up time of 46 days (IQR 15.75-63 days) between exams was observed. Upon five-fold cross-validation, the model demonstrated a 0.44 Dice score overlap between predicted and true areas of future GBM tumor growth. The high sensitivity model yielded a per-voxel sensitivity of 0.91 (IQR 0.77-0.99) and PPV of 0.26 (IQR 0.17 to 0.32), while the high PPV model yielded a per-voxel sensitivity of 0.14 (IQR 0.00 to 0.46) and PPV of 0.75 (IQR 0.56 to 0.94). Upon visual confirmation, model predictions across incremental time values for any given exam yielded expected gradual growth of tumor over time.

The technology disclosed illustrates that a deep learning model can accurately predict future areas of GBM tumor growth in patients receiving TTFields therapy, with optimal performance that may be calibrated for high sensitivity or high PPV based on clinical use case.

As the growth of GBM tumors are aggressive, the technology disclosed allows the physicians to predict the regions in which the tumor is expected to grow in future. This prediction, enables, the physicians to better plan the treatment of the patients in follow-up therapy sessions.

### System Overview

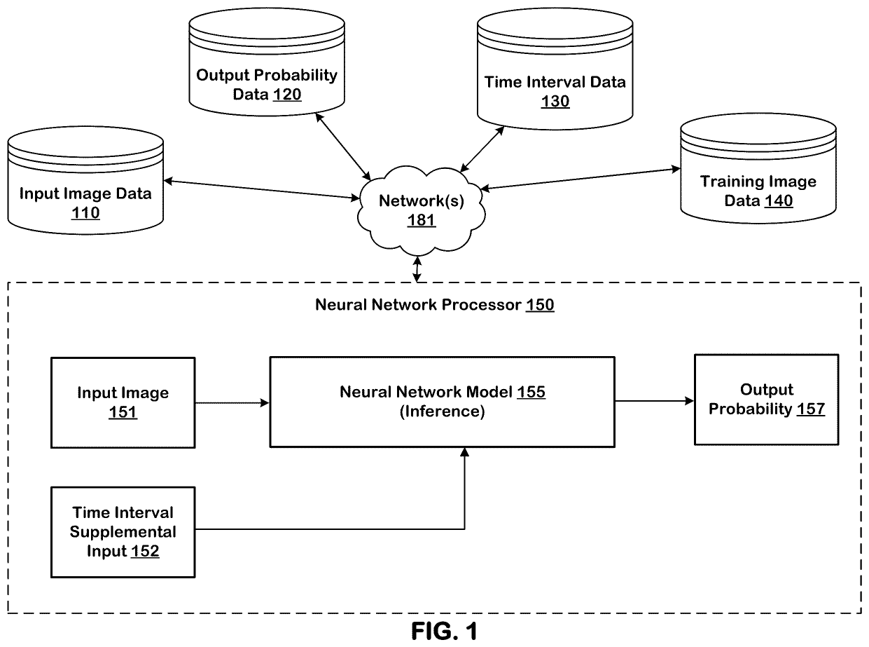

A system and various implementations of the technology to predict fluctuations in sea surface temperature and to predict extreme climate events is described with reference to FIGS. 1-14.

FIG. 1 illustrates an example architectural-level schematic of a system that uses a trained machine learning model to predict future growth (or spread) of GBM tumor on follow-up examinations given the precursor examination and time interval between precursor examination and the follow-up examination. Because FIG. 1 is an architectural diagram, certain details are omitted to improve the clarity of the description. The discussion of FIG. 1 is organized as follows. First, the elements of the system are described, followed by their interconnections. Then, the use of the elements in the system is described in greater detail.

Glioblastoma (GBM) is the most common type of malignant (cancerous) brain tumor that starts in the brain in adults. Tumor-treating fields, a noninvasive and innovative therapeutic approach. The TTF therapy is carried out in multiple sessions over many weeks and months. The technology disclosed uses a trained machine learning model to predict regions in which tumor growth is expected in future. FIG. 1 presents a system that can be used to predict the regions in which the tumor is expected to grow using the MRI images for the patient from the current TTF therapy session.

This paragraph names labeled parts of the system presented in FIG. 1. The system includes a neural network processor 150, an input image database 110, an output probability database 120, a time interval database 130 and a training image database 140, all in communication with each other via network(s) 181. The neural network processor 150 comprises one or more neural network models 155. FIG. 1, illustrates, the neural network model 155, operating in inference (or production) mode. In this mode, an input image 151 is provided as input to the neural network model 155. The neural network model 155 processes the input image data (151) to generate an output probability data 157. The neural network processor 150 determines the output probability 157 based in part on a time interval data 152 that is provided as a supplemental input to the neural network model 155.

In one implementation, the neural network model 150 is a convolutional neural network (CNN). A convolutional neural network (CNN) is a type of deep learning network that can process and analyze data like images, text, and audio. CNNs are often used for image recognition and computer vision tasks. Convolutional neural networks can have three types of layers including convolutional layer, pooling layer and fully connected layer. The convolutional layer is the first layer of a convolutional network. While convolutional layers can be followed by additional convolutional layers or pooling layers, the fully-connected layer is the final layer. With each layer, the CNN increases in its complexity, identifying greater portions of the image. Earlier layers focus on simple features, such as colors and edges. As the image data progresses through the layers of the CNN, it starts to recognize larger elements or shapes of the object. The convolutional layer is the core building block of a CNN, and it is where the majority of computation occurs. It requires a few components, which are input data, a filter and a feature map. The input image can be considered as a matrix of pixels in 3D (three dimensions). This means that the input will have three dimensions, a height, width and depth. The depth is also referred to as channels that correspond to RGB in an image. There is a feature detector, also known as a kernel or a filter, which will move across the receptive fields of the image, checking if the feature is present. This process is known as a convolution. Pooling layers, also known as downsampling, conducts dimensionality reduction, reducing the number of parameters in the input. Similar to the convolutional layer, the pooling operation sweeps a filter across the entire input, but the difference is that this filter does not have any weights. Instead, the kernel applies an aggregation function to the values within the receptive field, populating the output array. There are two main types of pooling, max pooling and average pooling. The pixel values of the input image are not directly connected to the output layer in partially connected layers that are the intermediate layers prior to the fully connected layer. In the fully-connected layer, each node in the output layer connects directly to a node in the previous layer. This layer performs the task of classification based on the features extracted through the previous layers and their different filters. For example, the trained neural network model 155 can classify a region of an image (for a future examination) as containing a tumor or it can classify the region of the image as healthy with no tumor. CNNs can take raw image data such as pixels as input and learn to extract features like shapes and textures from the image. During training, CNNs can use a backpropagation algorithm to learn spatial hierarchies of features. Convolutional neural networks can use artificial neurons to calculate the weighted sum of inputs and output an activation value.

The convolutional neural network can have an encoder-decoder architecture. An encoder-decoder CNN, or ED-CNN, is a specific type of CNN architecture that consists of two interconnected subnetworks: an encoder and a decoder. The encoder subnetwork takes an input image and compresses it into a lower-dimensional representation, also known as a latent space. This process involves passing the input image through multiple layers of convolution and pooling operations, which gradually reduce the spatial dimensions of the image while extracting important features. The resulting compressed representation is then passed on to the decoder subnetwork, which uses an inverse process to reconstruct the original image from the compressed representation. The decoder typically employs the same architecture as the encoder, but in reverse order, with upsampling and deconvolution operations instead of downsampling and convolution operations. ED-CNNs are often used in image-to-image regression tasks, where the goal is to learn a mapping between input and output images. By learning to encode the input image into a compressed representation and then decode it back into the output image, ED-CNNs can effectively learn complex and non-linear mappings between images while minimizing the number of parameters needed for the network. Further details of the encoder-decoder architecture are presented in a following section.

Some implementations of the technology disclosed relate to using a Transformer model to provide an AI system. In particular, the technology disclosed proposes a parallel input, parallel output (PIPO) AI system based on the Transformer architecture. The Transformer model relies on a self-attention mechanism to compute a series of context-informed vector-space representations of elements in the input sequence and the output sequence, which are then used to predict distributions over subsequent elements as the model predicts the output sequence element-by-element. Not only is this mechanism straightforward to parallelize, but as each input's representation is also directly informed by all other inputs' representations, this results in an effectively global receptive field across the whole input sequence. This stands in contrast to, e.g., convolutional architectures which typically only have a limited receptive field.

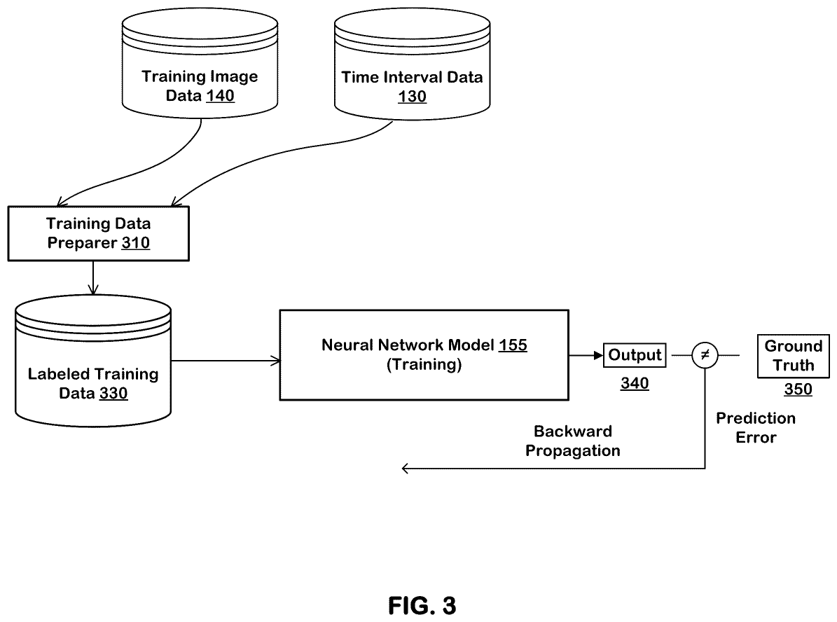

The technology disclosed uses time interval between a timestamp of the input image data representing current spatial distribution of the GBM detected at a precursor examination of a patient and a timestamp of a follow-up examination as a supplemental input (152) to the neural network model 155. In one implementation, the neural network processor 150 is configured to use a supplemental time feature channel to supply the neural network 155 (also referred to as the neural network model 155) with temporal information characterizing the time interval. The neural network processor 150 is configured with logic to concatenate the supplemental time feature channel with a penultimate feature map generated by the neural network. In one implementation, the neural network processor 150 is further configured with logic to cause the neural network to use a concatenation of the supplemental time feature channel and the penultimate feature map to generate the future spatial distribution. The concatenation allows the neural network to calibrate the future spatial distribution based on elapsed time between the precursor examination and the follow-up examination. In one implementation, the concatenation is a four-dimensional (4D) representation. In one implementation, the neural network 155 (or the neural network model 155) is trained using a binary cross-entropy loss function. In one implementation, the neural network is trained on training image data in which certain regions of enhancing tumor core are delineated and aligned across time points using nonlinear deformable registration. The training data images are stored in the training image database 140. Further details of the training are presented with reference to FIG. 3.

The output probability data 157 generated by the neural network model 155 therefore, characterizes a future spatial distribution of the GMB at the follow-up examination of the patient receiving the TTFields therapy. The neural network model is trained to predict the output probability 157 in dependence on the time elapsed between the timestamp of precursor examination of the patient and the timestamp of the follow-up examination. The output from the neural network model 155 therefore, allows the physicians to analyze the spread or growth of the tumor in advance. This information can be helpful in planning the follow-up TTFields therapy sessions as GBM is an aggressive type of tumor that can spread very quickly. In one implementation, the input image data 151 and the output probability data 157 are two-dimensional image data. In another implementation, the input image data 151 and the output probability data 157 are three-dimensional image data.

In one implementation, the input image data 151 and the output probability data 157 can be three-dimensional magnetic resonance imaging (MRI) data. In one implementation, the input image data is a voxel grid. A voxel grid geometry is a 3D grid of values organized into layers of rows and columns. Each row, column, and layer intersection in the grid is called a voxel or small 3D cube. A “voxel grid image data” refers to a 3D representation of an image where the data is organized into a grid of small cubic units called “voxels,” that can be considered as 3D pixels, where each voxel holds a value representing the intensity or color at that specific point in space. Voxel grids are often used to represent medical scans such as MRIs, where the data is represented as a 3D volume of tissue densities. In one implementation, the output probability 157, which represents the future spatial distribution, is represented by a dense per-voxel prediction of a future tumor growth probability for each voxel in the voxel grid. In one implementation, the future spatial distribution is represented by a heat map of probability scores.

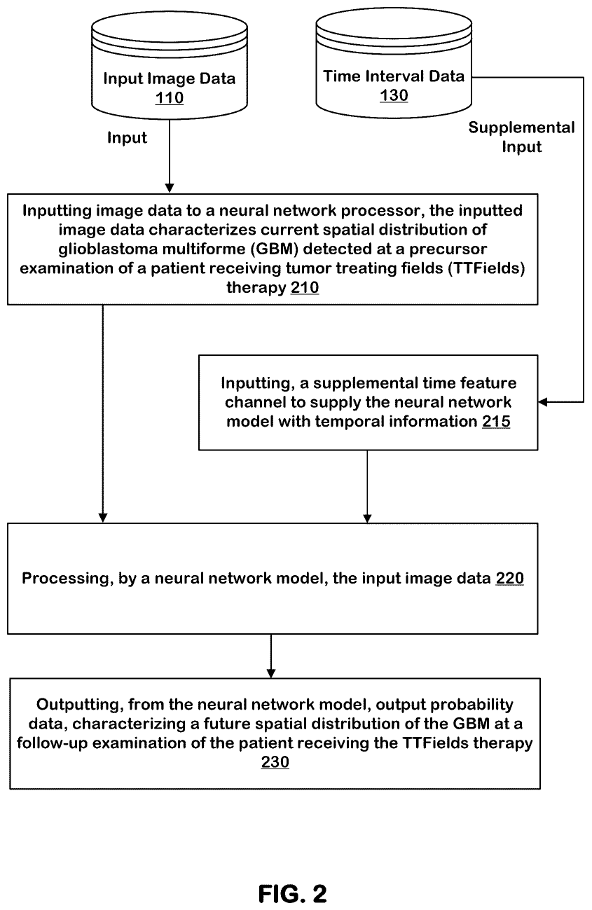

Completing the description of FIG. 1, the components of the system in FIG. 1, described above, are all coupled in communication with the network(s) 181. The actual communication path can be point-to-point over public and/or private networks. The communications can occur over a variety of networks, e.g., private networks, VPN, MPLS circuit, RFID, or Internet, and can use appropriate application programming interfaces (APIs) and data interchange formats, e.g., Representational State Transfer (REST), Electronic Data Interchange (EDI), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, satellite network, point-to-point network, star network, token ring network, hub network, Internet, inclusive of the mobile Internet, via protocols such as EDGE, 3G, 4G LTE, Wi-Fi and WiMAX. The engines, data processors or system components of FIG. 1 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications. In the following section, details of the method disclosed herein are presented with reference to the flowchart in FIG. 2.

### Process Flow Diagram

FIG. 2 presents an example process flow diagram comprising process operations to generate a characterization of a future spatial distribution of a GBM tumor at a follow-up examination of the patient receiving the TTFields therapy. The order of operations illustrated in the flow diagram in FIG. 2 is provided for the purposes of illustration, and can be modified to suit a particular implementation. Many of the operations, for example, can be executed in parallel. One or more operations in the flow diagram in FIG. 2 can be combined and performed together in a single operation. Similarly, one or more operations can be further divided into sub-operations that can be executed in parallel or in a serial manner.

The process flow diagram (or process flow chart) in FIG. 2 starts with inputting the image data to a trained neural network processor 150 comprising a neural network model 155 (operation 210). The inputted image data is received from the input image database 110. The inputted image data characterizes current spatial distribution of glioblastoma multiforme (GBM) detected at a precursor examination of a patient receiving tumor treating fields (TTFields or TTF) therapy. The neural network model processes the input image data at an operation 220. The method includes inputting a supplemental input to the neural network model. The supplemental input is a supplemental time feature channel to supply the neural network model with temporal information (operation 215). The supplemental input can be received from the time interval database 130. The neural network model 155 determines the output (i.e., the future spatial distribution) based in part on the time interval supplemental input. The time interval data identifies the time interval between the precursor examination and the follow-up examination. The neural network model 155 outputs (at an operation 230), the output probability data 157. The output probability data 157 characterizes a future spatial distribution of the GBM at a follow-up examination of the patient receiving the TTF (or TTFields) therapy.

### Training of the Machine Learning Model

FIG. 3 presents a high-level architecture for training the neural network model 155. A training data preparer 310 includes logic to prepare labeled training data that can be stored in a labeled training data database 330. The labeled training data includes images from the training image database 140 and time interval data from the time interval database 130. The labeled training data is provided as input the neural network model 155 when it operates in a training mode as shown in in FIG. 3. The training data preparer 310 includes logic to create examples of training data for storing in labeled training database 330 that includes labeled images and time intervals between precursor examinations and follow-up examinations. This training data is provided as input to the neural network model 155 during training. In the training image data certain regions of enhancing tumor core are delineated and aligned across time points using nonlinear deformable registration. The time interval data is provided as supplemental input. In one implementation, the training data comprises patient data from a single institutional database that is queried to identify adult patients with histologically confirmed newly diagnosed and/or recurrent GBM undergoing TTFields therapy. For each patient, all serial follow-up MRI examinations were obtained, including T1 (pre-/post-contrast), T2, and T2/FLAIR sequences. On each exam, all regions of enhancing tumor core (excluding peritumoral edema, necrotic core, or resection cavity) were delineated and aligned across time points using nonlinear deformable registration. For any given pair of serial examinations, the neural network model 155 (implemented as a convolutional neural network or CNN) is trained to predict future GBM tumor on the follow-up examination given the precursor examination and time interval between the studies (i.e., the precursor examination and the follow-up examination).

In one instance the labeled training data 330 included data collected from a total of 123 patients (1112 total MR exams). For any given single patient, a median of 6 follow-up exams (IQR 2.5-12.5) at a median interval follow-up time of 46 days (IQR 15.75-63 days) between examinations was observed. Upon five-fold cross-validation, the model demonstrated a 0.44 Dice score overlap between predicted and true areas of future GBM tumor growth. The high sensitivity model yielded a per-voxel sensitivity of 0.91 (IQR 0.77-0.99) and PPV of 0.26 (IQR 0.17 to 0.32), while the high PPV model yielded a per-voxel sensitivity of 0.14 (IQR 0.00 to 0.46) and PPV of 0.75 (IQR 0.56 to 0.94). Upon visual confirmation, model predictions across incremental time values for any given exam yielded expected gradual growth of tumor over time

During training, the output (340) from the machine learning model 155 is compared with ground truth labels (350) and a prediction error is calculated. During backward propagation, the weights of the machine learning model are adjusted to reduce the prediction error. The trained machine learning model is then used for processing production images. In one implementation, the neural network is trained using a binary cross-entropy loss function. Other loss or error prediction functions can be used such as categorical cross-entropy loss function, binary focal cross-entropy loss function, etc. Class weights are used to develop high sensitivity and high positive predictive value (PPV) model variants. To generate final logit scores, time information is concatenated to the penultimate feature map as an additional feature channel, allowing the model to calibrate each estimate based on elapsed time between any pair of exams. Upon convergence, a 4D learned representation allows for prediction of spatial distribution of GBM tumor at any future time point. The technology disclosed illustrates that a deep learning model can accurately predict future areas of GBM tumor growth in patients receiving TTFields therapy, with optimal performance that may be calibrated for high sensitivity or high PPV based on clinical use case.

In one implementation, the disclosed AI system is a multilayer perceptron (MLP). In another implementation, the disclosed AI system is a feedforward neural network. In yet another implementation, the disclosed AI system is a fully connected neural network. In a further implementation, the disclosed AI system is a fully convolution neural network. In a yet further implementation, the disclosed AI system is a semantic segmentation neural network. In a yet another further implementation, the disclosed AI system is a generative adversarial network (GAN) (e.g., CycleGAN, StyleGAN, pixelRNN, text-2-image, DiscoGAN, IsGAN). In a yet another implementation, the disclosed AI system includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, various ChatGPT versions, various LLaMA versions, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In one implementation, the disclosed AI system is a convolution neural network (CNN) with a plurality of convolution layers. In another implementation, the disclosed AI system is a recurrent neural network (RNN) such as a long short-term memory network (LSTM), bi-directional LSTM (Bi-LSTM), or a gated recurrent unit (GRU). In yet another implementation, the disclosed AI system includes both a CNN and an RNN.

In yet other implementations, the disclosed AI system can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The disclosed AI system can use one or more loss functions such as logistic regression/log loss, multi-class cross-entropy/softmax loss, binary cross-entropy loss, mean-squared error loss, L1 loss, L2 loss, smooth L1 loss, and Huber loss. The disclosed AI system can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The disclosed AI system can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

The disclosed AI system can be a linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The disclosed AI system can be an ensemble of multiple models, in some implementations.

In some implementations, the disclosed AI system can be trained using backpropagation-based gradient update techniques. Example gradient descent techniques that can be used for training the disclosed AI system include stochastic gradient descent, batch gradient descent, and mini-batch gradient descent. Some examples of gradient descent optimization algorithms that can be used to train the disclosed AI system are Momentum, Nesterov accelerated gradient, Adagrad, Adadelta, RMSprop, Adam, AdaMax, Nadam, and AMSGrad.

### Transformer Logic

Machine learning is the use and development of computer systems that can learn and adapt without following explicit instructions, by using algorithms and statistical models to analyze and draw inferences from patterns in data. Some of the state-of-the-art models use Transformers, a more powerful and faster model than neural networks alone. Transformers originate from the field of natural language processing (NLP), but can be used in computer vision and many other fields. Neural networks process input in series and weight relationships by distance in the series. Transformers can process input in parallel and do not necessarily weigh by distance. For example, in natural language processing, neural networks process a sentence from beginning to end with the weights of words close to each other being higher than those further apart. This leaves the end of the sentence very disconnected from the beginning causing an effect called the vanishing gradient problem. Transformers look at each word in parallel and determine weights for the relationships to each of the other words in the sentence. These relationships are called hidden states because they are later condensed for use into one vector called the context vector. Transformers can be used in addition to neural networks. This architecture is described here.

### Encoder-Decoder Architecture

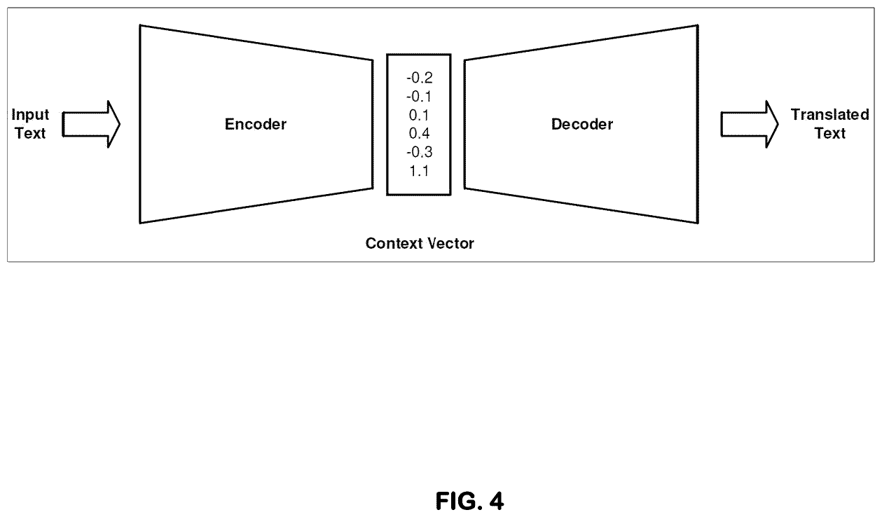

FIG. 4 is a schematic representation of an encoder-decoder architecture. This architecture is often used for NLP and has two main building blocks. The first building block is the encoder that encodes an input into a fixed-size vector. In the system we describe here, the encoder is based on a recurrent neural network (RNN). At each time step, t, a hidden state of time step, t-1, is combined with the input value at time step t to compute the hidden state at timestep t. The hidden state at the last time step, encoded in a context vector, contains relationships encoded at all previous time steps. For NLP, each step corresponds to a word. Then the context vector contains information about the grammar and the sentence structure. The context vector can be considered a low-dimensional representation of the entire input space. For NLP, the input space is a sentence, and a training set consists of many sentences.

The context vector is then passed to the second building block, the decoder. For translation, the decoder has been trained on a second language. Conditioned on the input context vector, the decoder generates an output sequence. At each time step, t, the decoder is fed the hidden state of time step, t-1, and the output generated at time step, t-1. The first hidden state in the decoder is the context vector, generated by the encoder. The context vector is used by the decoder to perform the translation.

The whole model is optimized end-to-end by using backpropagation, a method of training a neural network in which the initial system output is compared to the desired output and the system is adjusted until the difference is minimized. In backpropagation, the encoder is trained to extract the right information from the input sequence, the decoder is trained to capture the grammar and vocabulary of the output language. This results in a fluent model that uses context and generalizes well. When training an encoder-decoder model, the real output sequence is used to train the model to prevent mistakes from stacking. When testing the model, the previously predicted output value is used to predict the next one.

When performing a translation task using the encoder-decoder architecture, all information about the input sequence is forced into one vector, the context vector. Information connecting the beginning of the sentence with the end is lost, the vanishing gradient problem. Also, different parts of the input sequence are important for different parts of the output sequence, information that cannot be learned using only RNNs in an encoder-decoder architecture.

### Attention Mechanism

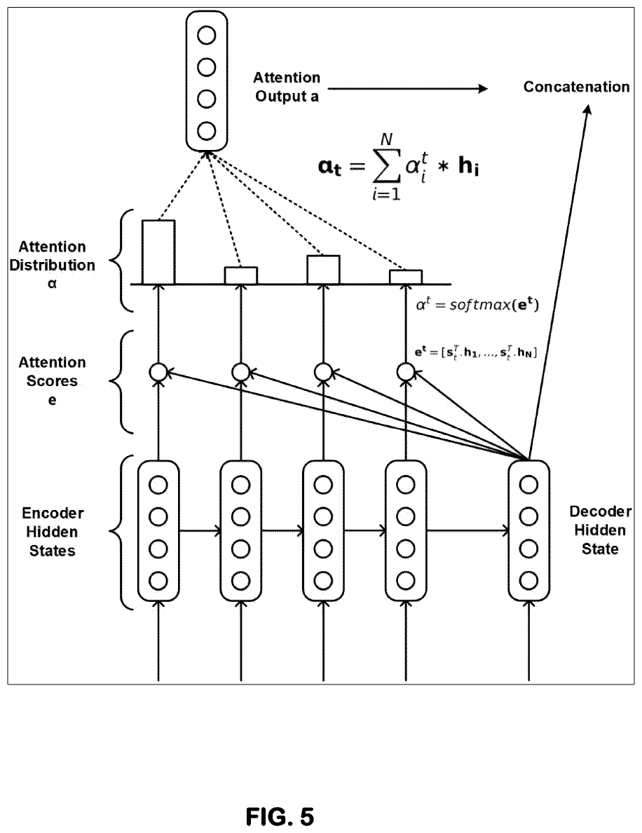

Attention mechanisms distinguish Transformers from other machine learning models. The attention mechanism provides a solution for the vanishing gradient problem. FIG. 5 shows an overview of an attention mechanism added onto an RNN encoder-decoder architecture. At every step, the decoder is given an attention score, e, for each encoder hidden state. In other words, the decoder is given weights for each relationship between words in a sentence. The decoder uses the attention score concatenated with the context vector during decoding. The output of the decoder at time step t is based on all encoder hidden states and the attention outputs. The attention output captures the relevant context for time step t from the original sentence. Thus, words at the end of a sentence may now have a strong relationship with words at the beginning of the sentence. In the sentence “The quick brown fox, upon arriving at the doghouse, jumped over the lazy dog,” fox and dog can be closely related despite being far apart in this complex sentence.

To weight encoder hidden states, a dot product between the decoder hidden state of the current time step, and all encoder hidden states, is calculated. This results in an attention score for every encoder hidden state. The attention scores are higher for those encoder hidden states that are similar to the decoder hidden state of the current time step. Higher values for the dot product indicate the vectors are pointing more closely in the same direction. The attention scores are converted to fractions that sum to one using the SoftMax function.

The SoftMax scores provide an attention distribution. The x-axis of the distribution is position in a sentence. The y-axis is attention weight. The scores show which encoder hidden states are most closely related. The SoftMax scores specify which encoder hidden states are the most relevant for the decoder hidden state of the current time step.

The elements of the attention distribution are used as weights to calculate a weighted sum over the different encoder hidden states. The outcome of the weighted sum is called the attention output. The attention output is used to predict the output, often in combination (concatenation) with the decoder hidden states. Thus, both information about the inputs, as well as the already generated outputs, can be used to predict the next outputs.

By making it possible to focus on specific parts of the input in every decoder step, the attention mechanism solves the vanishing gradient problem. By using attention, information flows more directly to the decoder. It does not pass through many hidden states. Interpreting the attention step can give insights into the data. Attention can be thought of as a soft alignment. The words in the input sequence with a high attention score align with the current target word. Attention describes long-range dependencies better than RNN alone. This enables analysis of longer, more complex sentences.

The attention mechanism can be generalized as: given a set of vector values and a vector query, attention is a technique to compute a weighted sum of the vector values, dependent on the vector query. The vector values are the encoder hidden states, and the vector query is the decoder hidden state at the current time step.

The weighted sum can be considered a selective summary of the information present in the vector values. The vector query determines on which of the vector values to focus. Thus, a fixed-size representation of the vector values can be created, in dependence upon the vector query.

The attention scores can be calculated by the dot product, or by weighing the different values (multiplicative attention).

### Embeddings

For most machine learning models, the input to the model needs to be numerical. The input to a translation model is a sentence, and words are not numerical. multiple methods exist for the conversion of words into numerical vectors. These numerical vectors are called the embeddings of the words. Embeddings can be used to convert any type of symbolic representation into a numerical one.

Embeddings can be created by using one-hot encoding. The one-hot vector representing the symbols has the same length as the total number of possible different symbols. Each position in the one-hot vector corresponds to a specific symbol. For example, when converting colors to a numerical vector, the length of the one-hot vector would be the total number of different colors present in the dataset. For each input, the location corresponding to the color of that value is one, whereas all the other locations are valued at zero. This works well for working with images. For NLP, this becomes problematic, because the number of words in a language is very large. This results in enormous models and the need for a lot of computational power. Furthermore, no specific information is captured with one-hot encoding. From the numerical representation, it is not clear that orange and red are more similar than orange and green. For this reason, other methods exist.

A second way of creating embeddings is by creating feature vectors. Every symbol has its specific vector representation, based on features. With colors, a vector of three elements could be used, where the elements represent the amount of yellow, red, and/or blue needed to create the color. Thus, all colors can be represented by only using a vector of three elements. Also, similar colors have similar representation vectors.

For NLP, embeddings based on context, as opposed to words, are small and can be trained. The reasoning behind this concept is that words with similar meanings occur in similar contexts. Different methods take the context of words into account. Some methods, like GloVe, base their context embedding on co-occurrence statistics from corpora (large texts) such as Wikipedia. Words with similar co-occurrence statistics have similar word embeddings. Other methods use neural networks to train the embeddings. For example, they train their embeddings to predict the word based on the context (Common Bag of Words), and/or to predict the context based on the word (Skip-Gram). Training these contextual embeddings is time intensive. For this reason, pre-trained libraries exist. Other deep learning methods can be used to create embeddings. For example, the latent space of a variational autoencoder (VAE) can be used as the embedding of the input. Another method is to use 1D convolutions to create embeddings. This causes a sparse, high-dimensional input space to be converted to a denser, low-dimensional feature space.

### Self-Attention: Queries (Q), Keys (K), Values (V)

Transformer models are based on the principle of self-attention. Self-attention allows each element of the input sequence to look at all other elements in the input sequence and search for clues that can help it to create a more meaningful encoding. It is a way to look at which other sequence elements are relevant for the current element. The Transformer can grab context from both before and after the currently processed element.

When performing self-attention, three vectors need to be created for each element of the encoder input: the query vector (Q), the key vector (K), and the value vector (V). These vectors are created by performing matrix multiplications between the input embedding vectors using three unique weight matrices.

After this, self-attention scores are calculated. When calculating self-attention scores for a given element, the dot products between the query vector of this element and the key vectors of all other input elements are calculated. To make the model mathematically more stable, these self-attention scores are divided by the root of the size of the vectors. This has the effect of reducing the importance of the scalar thus emphasizing the importance of the direction of the vector. Just as before, these scores are normalized with a SoftMax layer. This attention distribution is then used to calculate a weighted sum of the value vectors, resulting in a vector z for every input element. In the attention principle explained above, the vector to calculate attention scores and to perform the weighted sum was the same, in self-attention two different vectors are created and used. As the self-attention needs to be calculated for all elements (thus a query for every element), one formula can be created to calculate a Z matrix. The rows of this Z matrix are the z vectors for every sequence input element, giving the matrix a size length sequence dimension QKV.

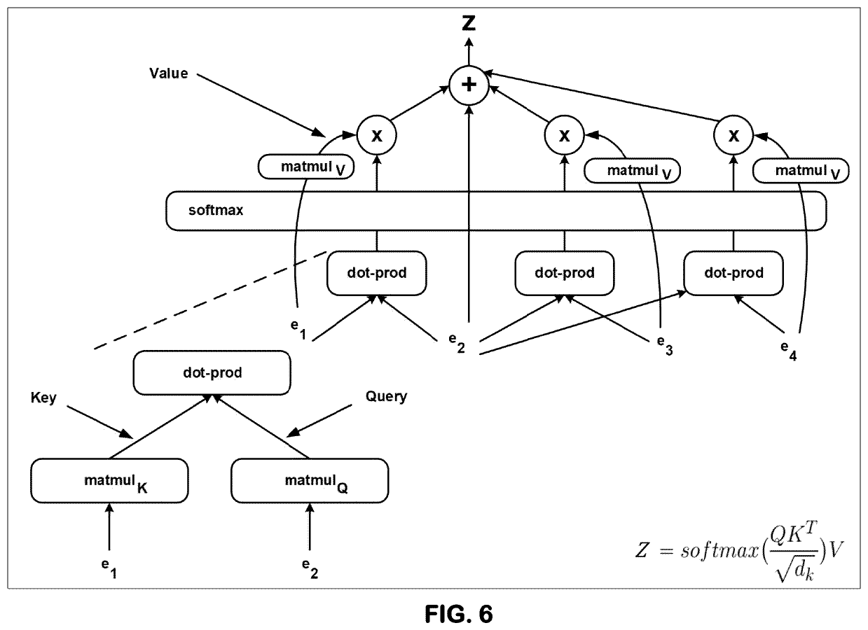

Multi-headed attention is executed in the Transformer. FIG. 6 is a schematic representation of the calculation of self-attention showing one attention head. For every attention head, different weight matrices are trained to calculate Q, K, and V. Every attention head outputs a matrix Z. Different attention heads can capture different types of information. The different Z matrices of the different attention heads are concatenated. This matrix can become large when multiple attention heads are used. To reduce dimensionality, an extra weight matrix W is trained to condense the different attention heads into a matrix with the same size as one Z matrix. This way, the amount of data given to the next step does not enlarge every time self-attention is performed.

When performing self-attention, information about the order of the different elements within the sequence is lost. To address this problem, positional encodings are added to the embedding vectors. Every position has its unique positional encoding vector. These vectors follow a specific pattern, which the Transformer model can learn to recognize. This way, the model can consider distances between the different elements.

As discussed above, in the core of self-attention are three objects: queries (Q), keys (K), and values (V). Each of these objects has an inner semantic meaning of their purpose. One can think of these as analogous to databases. We have a user-defined query of what the user wants to know. Then we have the relations in the database, i.e., the values which are the weights. More advanced database management systems create some apt representation of its relations to retrieve values more efficiently from the relations. This can be achieved by using indexes, which represent information about what is stored in the database. In the context of attention, indexes can be thought of as keys. So instead of running the query against values directly, the query is first executed on the indexes to retrieve where the relevant values or weights are stored. Lastly, these weights are run against the original values to retrieve data that is most relevant to the initial query.

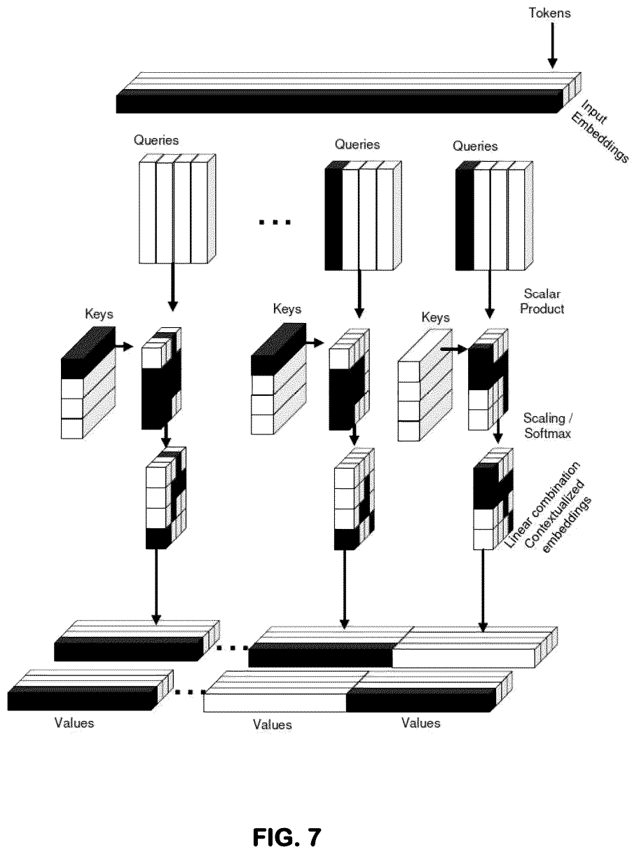

FIG. 7 depicts several attention heads in a Transformer block. We can see that the outputs of queries and keys dot products in different attention heads are differently colored. This depicts the capability of the multi-head attention to focus on different aspects of the input and aggregate the obtained information by multiplying the input with different attention weights.

Examples of attention calculation include scaled dot-product attention and additive attention. There are several reasons why scaled dot-product attention is used in the Transformers. Firstly, the scaled dot-product attention is relatively fast to compute, since its main parts are matrix operations that can be run on modern hardware accelerators. Secondly, it performs similarly well for smaller dimensions of the K matrix, dk, as the additive attention. For larger dk, the scaled dot-product attention performs a bit worse because dot products can cause the vanishing gradient problem. This is compensated via the scaling factor, which is defined as √dk.

As discussed above, the attention function takes as input three objects: key, value, and query. In the context of Transformers, these objects are matrices of shapes (n, d), where n is the number of elements in the input sequence and d is the hidden representation of each element (also called the hidden vector). Attention is then computed as:

$$Attention ( Q , K , V ) = SoftMax ( ( {QK}^{⋀} T ) / √ dk ) V$$

where Q, K, V are computed as:

X·W_Q,X·W_K,X·W_V

X is the input matrix and WQ, WK, WV are learned weights to project the input matrix into the representations. The dot products appearing in the attention function are exploited for their geometrical interpretation where higher values of their results mean that the inputs are more similar, i.e., pointing in the geometrical space in the same direction. Since the attention function now works with matrices, the dot product becomes matrix multiplication. The SoftMax function is used to normalize the attention weights into the value of 1 prior to being multiplied by the values matrix. The resulting matrix is used either as input into another layer of attention or becomes the output of the Transformer.

### Multi-Head Attention

Transformers become even more powerful when multi-head attention is used. Queries, keys, and values are computed the same way as above, though they are now projected into h different representations of smaller dimensions using a set of h learned weights. Each representation is passed into a different scaled dot-product attention block called a head. The head then computes its output using the same procedure as described above.

Formally, the multi-head attention is defined as:

MultiHeadAttention(Q,K,V)=[headl, . . . ,headh]W0 where headi=Attention (QW_i\hat{ }Q,KW_i\hat{ }K,VW_i\hat{ }V)

The outputs of all heads are concatenated together and projected again using the learned weights matrix W0 to match the dimensions expected by the next block of heads or the output of the Transformer. Using the multi-head attention instead of the simpler scaled dot-product attention enables Transformers to jointly attend to information from different representation subspaces at different positions.

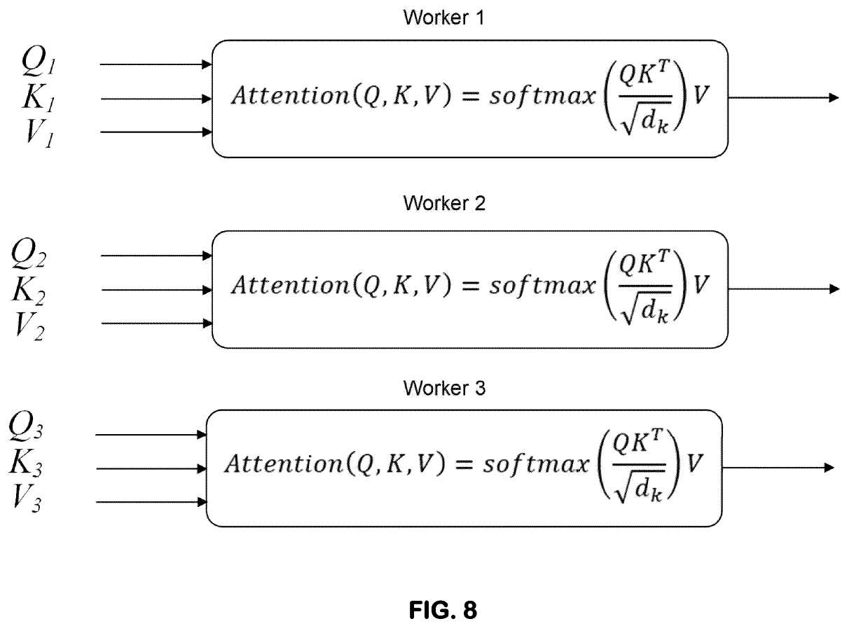

As shown in FIG. 8, one can use multiple workers to compute the multi-head attention in parallel, as the respective heads compute their outputs independently of one another. Parallel processing is one of the advantages of Transformers over RNNs.

Assuming the naive matrix multiplication algorithm which has a complexity of:

a·b·c

For matrices of shape (a, b) and (c, d), to obtain values Q, K, V, we need to compute the operations:

X·WQ,X·WK,X·WV

The matrix X is of shape (n, d) where n is the number of patches and d is the hidden vector dimension. The weights WQ, WK, WV are all of shape (d, d). Omitting the constant factor 3, the resulting complexity is:

n·d2

We can proceed to the estimation of the complexity of the attention function itself, i.e., of

SoftMax ((QK\hat{ }T)/√dk)V. The matrices Q and K are both of shape (n, d). The transposition operation does not influence the asymptotic complexity of computing the dot product of matrices of shapes (n, d)·(d, n), therefore its complexity is:

n2·d

Scaling by a constant factor of √dk, where dk is the dimension of the keys vector, as well as applying the SoftMax function, both have the complexity of a·b for a matrix of shape (a, b), hence they do not influence the asymptotic complexity. Lastly the dot product SoftMax ((QK\hat{ }T)/√dk)·V is between matrices of shapes (n, n) and (n, d) and so its complexity is:

n2·d

The final asymptotic complexity of scaled dot-product attention is obtained by summing the complexities of computing Q, K, V, and of the following attention function:

$$n \cdot d 2 + n 2 \cdot d .$$

The asymptotic complexity of multi-head attention is the same since the original input matrix X is projected into h matrices of shapes (n, d/h), where h is the number of heads. From the point of view of asymptotic complexity, h is constant, therefore we would arrive at the same estimate of asymptotic complexity using a similar approach as for the scaled dot-product attention.

Transformer models often have the encoder-decoder architecture, although this is not necessarily the case. The encoder is built out of different encoder layers which are all constructed in the same way. The positional encodings are added to the embedding vectors. Afterward, self-attention is performed.

### Encoder Block of Transformer

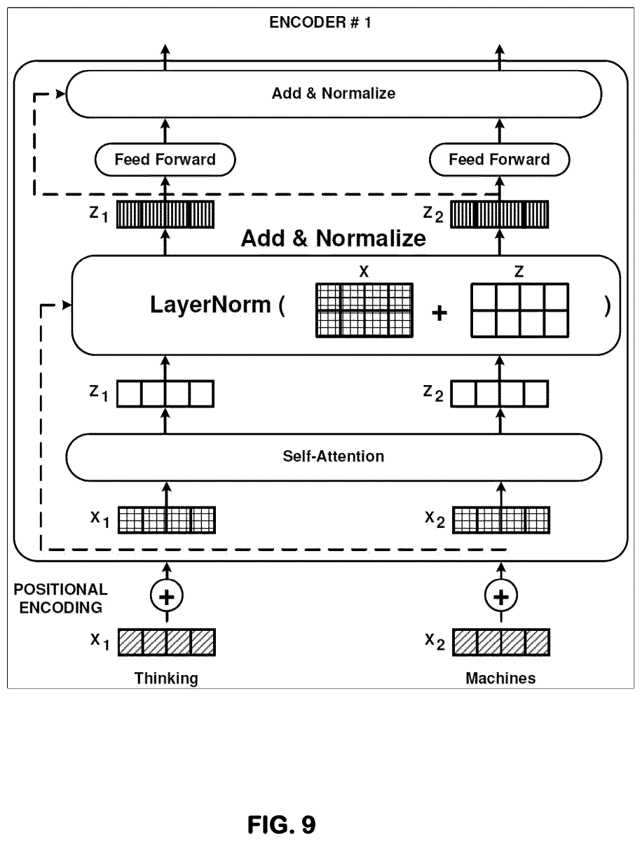

FIG. 9 portrays one encoder layer of a Transformer network. Every self-attention layer is surrounded by a residual connection, summing up the output and input of the self-attention. This sum is normalized, and the normalized vectors are fed to a feed-forward layer. Every z vector is fed separately to this feed-forward layer. The feed-forward layer is wrapped in a residual connection and the outcome is normalized too. Often, numerous encoder layers are piled to form the encoder. The output of the encoder is a fixed-size vector for every element of the input sequence.

Just like the encoder, the decoder is built from different decoder layers. In the decoder, a modified version of self-attention takes place. The query vector is only compared to the keys of previous output sequence elements. The elements further in the sequence are not known yet, as they still must be predicted. No information about these output elements may be used.

### Encoder-Decoder Blocks of Transformer

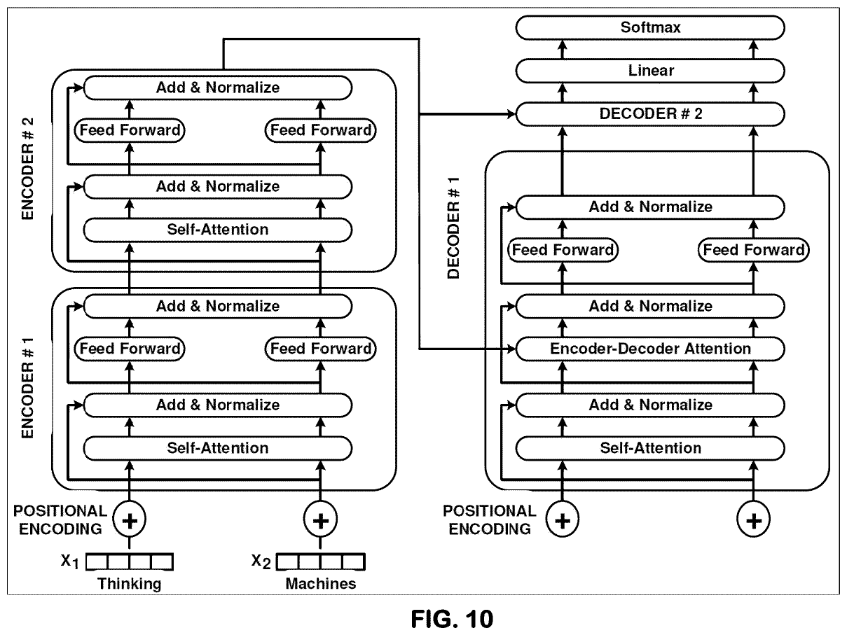

FIG. 10 shows a schematic overview of a Transformer model. Next to a self-attention layer, a layer of encoder-decoder attention is present in the decoder, in which the decoder can examine the last Z vectors of the encoder, providing fluent information transmission. The ultimate decoder layer is a feed-forward layer. All layers are packed in a residual connection. This allows the decoder to examine all previously predicted outputs and all encoded input vectors to predict the next output. Thus, information from the encoder is provided to the decoder, which could improve the predictive capacity. The output vectors of the last decoder layer need to be processed to form the output of the entire system. This is done by a combination of a feed-forward layer and a SoftMax function. The output corresponding to the highest probability is the predicted output value for a subject time step.

For some tasks other than translation, only an encoder is needed. This is true for both document classification and name entity recognition. In these cases, the encoded input vectors are the input of the feed-forward layer and the SoftMax layer. Transformer models have been extensively applied in different NLP fields, such as translation, document summarization, speech recognition, and named entity recognition. These models have applications in the field of biology as well for predicting protein structure and function and labeling DNA sequences.

### Vision Transformer

There are extensive applications of transformers in vision including popular recognition tasks (e.g., image classification, object detection, action recognition, and segmentation), generative modeling, multi-modal tasks (e.g., visual-question answering, visual reasoning, and visual grounding), video processing (e.g., activity recognition, video forecasting), low-level vision (e.g., image super-resolution, image enhancement, and colorization) and 3D analysis (e.g., point cloud classification and segmentation).

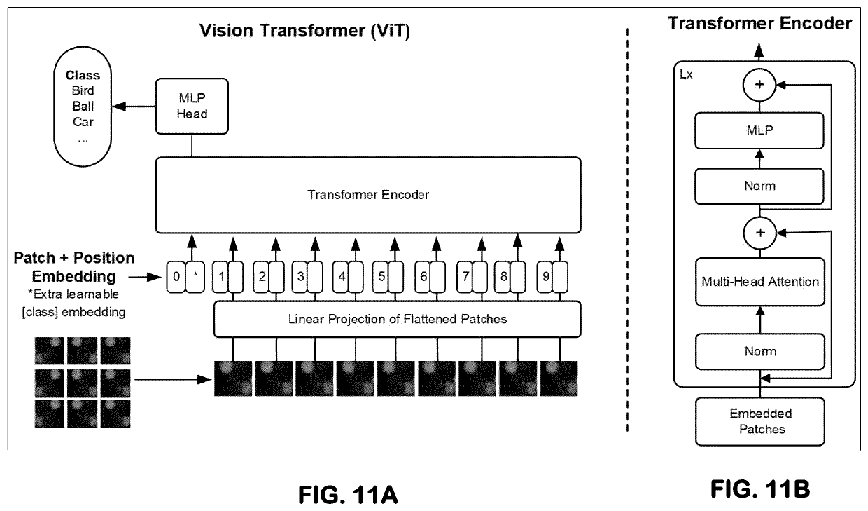

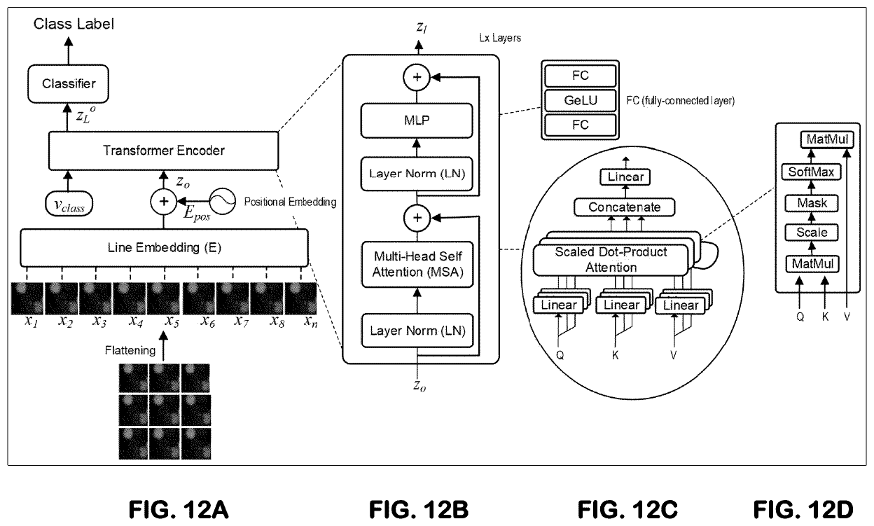

Transformers were originally developed for NLP and worked with sequences of words. In image classification, we often have a single input image in which the pixels are in a sequence. To reduce the computation required, Vision Transformers (ViTs) cut the input image into a set of fixed-sized patches of pixels. The patches are often 16×16 pixels. They are treated much like words in NLP Transformers. ViTs are depicted in FIGS. 11A, 11B, 12A, 12B, 12C, and 12D. Unfortunately, important positional information is lost because image sets are position-invariant. This problem is solved by adding a learned positional encoding into the image patches.

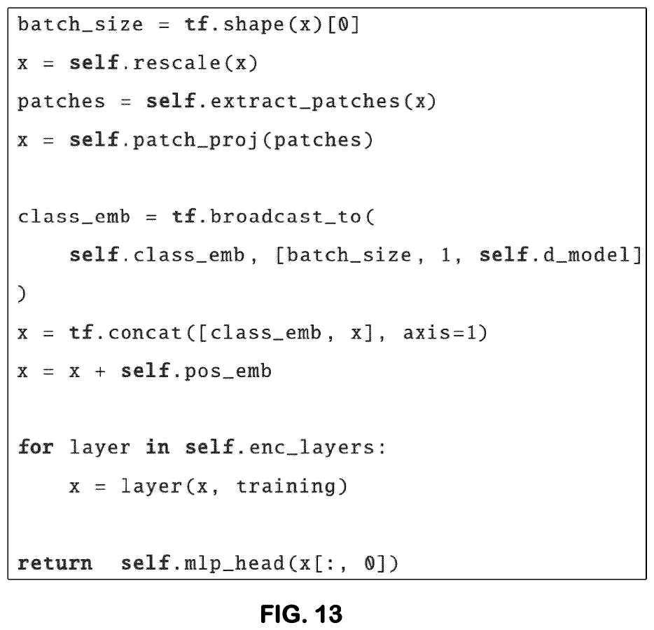

The computations of the ViT architecture can be summarized as follows. The first layer of a ViT extracts a fixed number of patches from an input image (FIG. 12A). The patches are then projected to linear embeddings. A special class token vector is added to the sequence of embedding vectors to include all representative information of all tokens through the multi-layer encoding procedure. The class vector is unique to each image. Vectors containing positional information are combined with the embeddings and the class token. The sequence of embedding vectors is passed into the Transformer blocks. The class token vector is extracted from the output of the last Transformer block and is passed into a multilayer perceptron (MLP) head whose output is the final classification. The perceptron takes the normalized input and places the output in categories. It classifies the images. This procedure directly translates into the Python Keras code shown in FIG. 13.

When the input image is split into patches, a fixed patch size is specified before instantiating a ViT. Given the quadratic complexity of attention, patch size has a large effect on the length of training and inference time. A single Transformer block comprises several layers. The first layer implements Layer Normalization, followed by the multi-head attention that is responsible for the performance of ViTs. In the depiction of a Transformer block in FIG. 8B, we can see two arrows. These are residual skip connections. Including skip connection data can simplify the output and improve the results. The output of the multi-head attention is followed again by Layer Normalization. And finally, the output layer is an MLP (Multi-Layer Perceptron) with the GELU (Gaussian Error Linear Unit) activation function.

ViTs can be pretrained and fine-tuned. Pretraining is generally done on a large dataset. Fine-tuning is done on a domain specific dataset.

Domain-specific architectures, like convolutional neural networks (CNNs) or long short-term memory networks (LSTMs), have been derived from the usual architecture of MLPs and suffer from so-called inductive biases that predispose the networks towards a certain output. ViTs stepped in the opposite direction of CNNs and LSTMs and became more general architectures by eliminating inductive biases. A ViT can be seen as a generalization of MLPs because MLPs, after being trained, do not change their weights for different inputs. On the other hand, ViTs compute their attention weights at runtime based on the particular input.

### Computer System

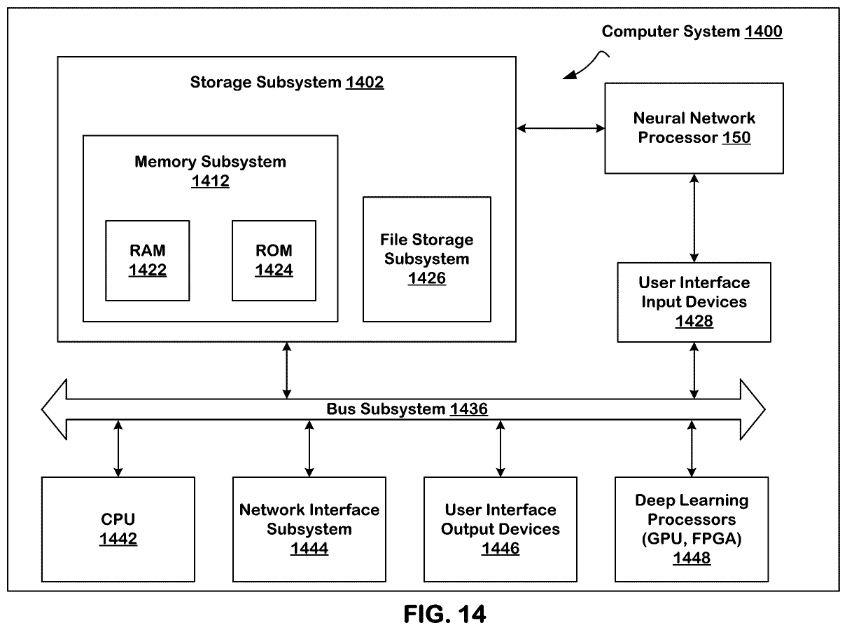

FIG. 14 shows an example computer system 1400 that can be used to implement the technology disclosed. Computer system 1400 includes at least one central processing unit (CPU) 1442 that communicates with a number of peripheral devices via bus subsystem 1436. These peripheral devices can include a storage subsystem 1402 including, for example, memory devices and a file storage subsystem 1426, user interface input devices 1428, user interface output devices 1446, and a network interface subsystem 1444. The input and output devices allow user interaction with computer system 1400. Network interface subsystem 1444 provides an interface to outside networks, including an interface to corresponding interface devices in other computer systems.

In one implementation, a neural network processor 150 is communicably linked to the storage subsystem 1402 and the user interface input devices 1428.

User interface input devices 1428 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1400.

User interface output devices 1446 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1400 to the user or to another machine or computer system.

Storage subsystem 1402 stores programming and data constructs that provide the functionality of some or all of the modules and methods described herein. These software modules are generally executed by processors 1448.

Processors 1448 can be graphics processing units (GPUs), field-programmable gate arrays (FPGAs), application-specific integrated circuits (ASICs), and/or coarse-grained reconfigurable architectures (CGRAs). Processors 1448 can be hosted by a deep learning cloud platform such as Google Cloud Platform™, Xilinx™, and Cirrascale™. Examples of processors 1148 include Google's Tensor Processing Unit (TPU)™, rackmount solutions like GX4 Rackmount Series™, GX11 Rackmount Series™, NVIDIA DGX-1™, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 1412 used in the storage subsystem 1402 can include a number of memories including a main random access memory (RAM) 1422 for storage of instructions and data during program execution and a read only memory (ROM) 1424 in which fixed instructions are stored. A file storage subsystem 1426 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1426 in the storage subsystem 1402, or in other machines accessible by the processor.

Bus subsystem 1436 provides a mechanism for letting the various components and subsystems of computer system 1400 communicate with each other as intended. Although bus subsystem 1436 is shown schematically as a single bus, alternative implementations of the bus subsystem can use multiple busses.

Computer system 1400 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 1400 depicted in FIG. 14 is intended only as a specific example for purposes of illustrating the preferred implementations of the present technology disclosed. Many other configurations of computer system 1400 are possible having more or less components than the computer system depicted in FIG. 14.

In various implementations, a learning system is provided. In some implementations, a feature vector is provided to a learning system. Based on the input features, the learning system generates one or more outputs. In some implementations, the output of the learning system is a feature vector. In some implementations, the learning system comprises an SVM. In other implementations, the learning system comprises an artificial neural network. In some implementations, the learning system is pre-trained using training data. In some implementations training data is retrospective data. In some implementations, the retrospective data is stored in a data store. In some implementations, the learning system may be additionally trained through manual curation of previously generated outputs.

In some implementations, an object detection pipeline is a trained classifier. In some implementations, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

FIG. 14 is a schematic of an exemplary computing node. Computing node 1400 is only one example of a suitable computing node and is not intended to suggest any limitation as to the scope of use or functionality of embodiments described herein. Regardless, computing node 1400 is capable of being implemented and/or performing any of the functionality set forth hereinabove.

In computing node 1400 there is a computer system/server, which is operational with numerous other general purpose or special purpose computing system environments or configurations. Examples of well-known computing systems, environments, and/or configurations that may be suitable for use with computer system/server include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed computing environments that include any of the above systems or devices, and the like.

Computer system/server may be described in the general context of computer system-executable instructions, such as program modules, being executed by a computer system. Generally, program modules may include routines, programs, objects, components, logic, data structures, and so on that perform particular tasks or implement particular abstract data types. Computer system/server may be practiced in distributed computing environments where tasks are performed by remote processing devices that are linked through a communications network. In a distributed computing environment, program modules may be located in both local and remote computer system storage media including memory storage devices.

As shown in FIG. 14, computer system/server in computing node 1400 is shown in the form of a general-purpose computing device. The components of computer system/server may include, but are not limited to, one or more processors or processing units, a system memory, and a bus that couples various system components including system memory to processor.

The bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server typically includes a variety of computer system readable media. Such media may be any available media that is accessible by computer system/server, and it includes both volatile and non-volatile media, removable and non-removable media.

System memory can include computer system readable media in the form of volatile memory, such as random access memory (RAM) and/or cache memory. Algorithm Computer system/server may further include other removable/non-removable, volatile/non-volatile computer system storage media. By way of example only, storage system can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus by one or more data media interfaces. As will be further depicted and described below, memory may include at least one program product having a set (e.g., at least one) of program modules that are configured to carry out the functions of embodiments of the disclosure.

Program/utility, having a set (at least one) of program modules, may be stored in memory by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments as described herein.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various implementations of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Clauses

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

1. A system, comprising:
memory storing input image data characterizing a current spatial distribution of glioblastoma multiforme (GBM), wherein the current spatial distribution of the GBM is detected at a precursor examination of a patient receiving tumor treating fields (TTFields) therapy; and
a neural network processor, in communication with the memory, and configured to cause a neural network to process the input image data and, in response, generate output probability data characterizing a future spatial distribution of the GBM at a follow-up examination of the patient receiving the TTFields therapy,
   wherein the neural network determines the future spatial distribution based in part on a time interval between the precursor examination and the follow-up examination.
      2. The system of clause 1, further configured to use the future spatial distribution to predict a future tumor growth of the GBM at the follow-up examination.
      3. The system of clause 1, wherein the neural network is a convolutional neural network.
      4. The system of clause 3, wherein the convolutional neural network has an encoder-decoder architecture.
      5. The system of clause 4, wherein the encoder-decoder architecture is a three-dimensional (3D) encoder-decoder architecture.
      6. The system of clause 1, wherein the input image data and the output probability data are two-dimensional (2D) image data.
      7. The system of clause 1, wherein the input image data and the output probability data are 3D image data.
      8. The system of clause 7, wherein the input image data and the output probability data are 3D magnetic resonance imaging (MRI) data.
      9. The system of clause 8, wherein the input image data is a voxel grid, and the future spatial distribution is represented by a dense per-voxel prediction of a future tumor growth probability for each voxel in the voxel grid.
      10. The system of clause 8, wherein the future spatial distribution is represented by a heat map of probability scores.
      11. The system of clause 1, further configured to use a supplemental time feature channel to supply the neural network with temporal information characterizing the time interval.
      12. The system of clause 11, further configured to concatenate the supplemental time feature channel with a penultimate feature map generated by the neural network.
      13. The system of clause 12, further configured to cause the neural network to use the concatenation of the supplemental time feature channel and the penultimate feature map to generate the future spatial distribution.
      14. The system of clause 13, wherein the concatenation allows the neural network to calibrate the future spatial distribution based on elapsed time between the precursor examination and the follow-up examination.
      15. The system of clause 13, wherein the concatenation is a four-dimensional (4D) representation.
      16. The system of clause 1, wherein the neural network is trained using a binary cross-entropy loss function.
      17. The system of clause 16, wherein the neural network is trained on training image data in which certain regions of enhancing tumor core are delineated and aligned across time points using nonlinear deformable registration.
      18. A method, including:
inputting, to a neural network processor, input image data characterizing a current spatial distribution of glioblastoma multiforme (GBM), wherein the current spatial distribution of the GBM is detected at a precursor examination of a patient receiving tumor treating fields (TTFields) therapy; and
processing the input image data using the neural network processor and, in response, generating output probability data characterizing a future spatial distribution of the GBM at a follow-up examination of the patient receiving the TTFields therapy,
   wherein the neural network determines the future spatial distribution based in part on a time interval between the precursor examination and the follow-up examination.
      19. The method of clause 18, further including, using the future spatial distribution to predict a future tumor growth of the GBM at the follow-up examination.
      20. The method of clause 18, wherein the neural network is a convolutional neural network.
      21. The method of clause 20, wherein the convolutional neural network has an encoder-decoder architecture.
      22. The method of clause 21, wherein the encoder-decoder architecture is a three-dimensional (3D) encoder-decoder architecture.
      23. The method of clause 18, wherein the input image data and the output probability data are two-dimensional (2D) image data.
      24. The method of clause 18, wherein the input image data and the output probability data are 3D image data.
      25. The method of clause 24, wherein the input image data and the output probability data are 3D magnetic resonance imaging (MRI) data.
      26. The method of clause 25, wherein the input image data is a voxel grid, and the future spatial distribution is represented by a dense per-voxel prediction of a future tumor growth probability for each voxel in the voxel grid.
      27. The method of clause 25, wherein the future spatial distribution is represented by a heat map of probability scores.
      28. The method of clause 18, further including, using a supplemental time feature channel to supply the neural network with temporal information characterizing the time interval.
      29. The method of clause 28, further including, concatenating the supplemental time feature channel with a penultimate feature map generated by the neural network.
      30. The method of clause 29, further including, causing the neural network to use the concatenation of the supplemental time feature channel and the penultimate feature map to generate the future spatial distribution.
      31. The method of clause 30, wherein the concatenation allows the neural network to calibrate the future spatial distribution based on elapsed time between the precursor examination and the follow-up examination.
      32. The method of clause 30, wherein the concatenation is a four-dimensional (4D) representation.
      33. The method of clause 18, wherein the neural network is trained using a binary cross-entropy loss function.
      34. The method of clause 33, wherein the neural network is trained on training image data in which certain regions of enhancing tumor core are delineated and aligned across time points using nonlinear deformable registration.
      35. A non-transitory computer readable storage medium impressed with computer program instructions, the instructions, when executed on a processor, implement a method, comprising:
inputting, to a neural network processor, input image data characterizing a current spatial distribution of glioblastoma multiforme (GBM), wherein the current spatial distribution of the GBM is detected at a precursor examination of a patient receiving tumor treating fields (TTFields) therapy; and
processing the input image data using the neural network processor and, in response, generating output probability data characterizing a future spatial distribution of the GBM at a follow-up examination of the patient receiving the TTFields therapy,
   wherein the neural network determines the future spatial distribution based in part on a time interval between the precursor examination and the follow-up examination.
      36. The non-transitory computer readable storage medium of clause 35, further configured to use the future spatial distribution to predict a future tumor growth of the GBM at the follow-up examination.
      37. The non-transitory computer readable storage medium of clause 35, wherein the neural network is a convolutional neural network.
      38. The non-transitory computer readable storage medium of clause 37, wherein the convolutional neural network has an encoder-decoder architecture.
      39. The non-transitory computer readable storage medium of clause 38, wherein the encoder-decoder architecture is a three-dimensional (3D) encoder-decoder architecture.
      40. The non-transitory computer readable storage medium of clause 35, wherein the input image data and the output probability data are two-dimensional (2D) image data.
      41. The non-transitory computer readable storage medium of clause 35, wherein the input image data and the output probability data are 3D image data.
      42. The non-transitory computer readable storage medium of clause 41, wherein the input image data and the output probability data are 3D magnetic resonance imaging (MRI) data.
      43. The non-transitory computer readable storage medium of clause 42, wherein the input image data is a voxel grid, and the future spatial distribution is represented by a dense per-voxel prediction of a future tumor growth probability for each voxel in the voxel grid.
      44. The non-transitory computer readable storage medium of clause 42, wherein the future spatial distribution is represented by a heat map of probability scores.
      45. The non-transitory computer readable storage medium of clause 35, implementing the method further comprising, using a supplemental time feature channel to supply the neural network with temporal information characterizing the time interval.
      46. The non-transitory computer readable storage medium of clause 45, implementing the method further comprising, concatenating the supplemental time feature channel with a penultimate feature map generated by the neural network.
      47. The non-transitory computer readable storage medium of clause 46, implementing the method further comprising, causing the neural network to use the concatenation of the supplemental time feature channel and the penultimate feature map to generate the future spatial distribution.
      48. The non-transitory computer readable storage medium of clause 47, wherein the concatenation allows the neural network to calibrate the future spatial distribution based on elapsed time between the precursor examination and the follow-up examination.
      49. The non-transitory computer readable storage medium of clause 47, wherein the concatenation is a four-dimensional (4D) representation.
      50. The non-transitory computer readable storage medium of clause 35, wherein the neural network is trained using a binary cross-entropy loss function.
      51. The non-transitory computer readable storage medium of clause 50, wherein the neural network is trained on training image data in which certain regions of enhancing tumor core are delineated and aligned across time points using nonlinear deformable registration.

What is claimed is:

1. A system, comprising:
memory storing input image data characterizing a current spatial distribution of glioblastoma multiforme (GBM), wherein the current spatial distribution of the GBM is detected at a precursor examination of a patient receiving tumor treating fields (TTFields) therapy; and
a neural network processor, in communication with the memory, and configured to cause a neural network to process the input image data and, in response, generate output probability data characterizing a future spatial distribution of the GBM at a follow-up examination of the patient receiving the TTFields therapy,
wherein the neural network determines the future spatial distribution based in part on a time interval between the precursor examination and the follow-up examination.

2. The system of claim 1, further configured to use the future spatial distribution to predict a future tumor growth of the GBM at the follow-up examination.

3. The system of claim 1, wherein the neural network is a convolutional neural network.

4. The system of claim 3, wherein the convolutional neural network has an encoder-decoder architecture.

5. The system of claim 4, wherein the encoder-decoder architecture is a three-dimensional (3D) encoder-decoder architecture.

6. The system of claim 1, wherein the input image data and the output probability data are two-dimensional (2D) image data.

7. The system of claim 1, wherein the input image data and the output probability data are 3D image data.

8. The system of claim 7, wherein the input image data and the output probability data are 3D magnetic resonance imaging (MRI) data.

9. The system of claim 8, wherein the input image data is a voxel grid, and the future spatial distribution is represented by a dense per-voxel prediction of a future tumor growth probability for each voxel in the voxel grid.

10. The system of claim 8, wherein the future spatial distribution is represented by a heat map of probability scores.

11. The system of claim 1, further configured to use a supplemental time feature channel to supply the neural network with temporal information characterizing the time interval.

12. The system of claim 11, further configured to concatenate the supplemental time feature channel with a penultimate feature map generated by the neural network.

13. The system of claim 12, further configured to cause the neural network to use the concatenation of the supplemental time feature channel and the penultimate feature map to generate the future spatial distribution.

14. The system of claim 13, wherein the concatenation allows the neural network to calibrate the future spatial distribution based on elapsed time between the precursor examination and the follow-up examination.

15. The system of claim 13, wherein the concatenation is a four-dimensional (4D) representation.

16. The system of claim 1, wherein the neural network is trained using a binary cross-entropy loss function.

17. The system of claim 16, wherein the neural network is trained on training image data in which certain regions of enhancing tumor core are delineated and aligned across time points using nonlinear deformable registration.

18. A method, including:
inputting, to a neural network processor, input image data characterizing a current spatial distribution of glioblastoma multiforme (GBM), wherein the current spatial distribution of the GBM is detected at a precursor examination of a patient receiving tumor treating fields (TTFields) therapy; and
processing the input image data using the neural network processor and, in response, generating output probability data characterizing a future spatial distribution of the GBM at a follow-up examination of the patient receiving the TTFields therapy,
wherein the neural network determines the future spatial distribution based in part on a time interval between the precursor examination and the follow-up examination.

19. The method of claim 18, further including, using the future spatial distribution to predict a future tumor growth of the GBM at the follow-up examination.

20. A non-transitory computer readable storage medium impressed with computer program instructions, the instructions, when executed on a processor, implement a method, comprising:
inputting, to a neural network processor, input image data characterizing a current spatial distribution of glioblastoma multiforme (GBM), wherein the current spatial distribution of the GBM is detected at a precursor examination of a patient receiving tumor treating fields (TTFields) therapy; and
processing the input image data using the neural network processor and, in response, generating output probability data characterizing a future spatial distribution of the GBM at a follow-up examination of the patient receiving the TTFields therapy,
wherein the neural network determines the future spatial distribution based in part on a time interval between the precursor examination and the follow-up examination.
